(19) United States
(12) Patent Application Publication
(10) Pub. No.: US 2025/0064321 A1
(43) Pub. Date: Feb. 27, 2025

(54) PHOTOACOUSTIC WAVE MEASURING DEVICE, PHOTOACOUSTIC WAVE MEASURING SYSTEM, AND THERMAL LIGHT SOURCE

(71) Applicant: TOYAMA PREFECTURAL UNIVERSITY, Imizu-shi, Toyama (JP)

(72) Inventors: Isao SHIMOYAMA, Imizu-shi, Toyama (JP); Kentaro NODA, Imizu-shi, Toyama (JP); Takuya TSUKAGOSHI, Imizu-shi, Toyama (JP)

(21) Appl. No.: 18/721,079

(22) PCT Filed: Dec. 23, 2022
(86) PCT No.: PCT/JP2022/047731

§ 371 Date: Sep. 20, 2024

(30) Foreign Application Priority Data
Dec. 24, 2021 (JP) 2021-211733

(51) Int. Cl. A61B 5/00

(52) U.S. Cl. CPC A61B 5/0095

(57) ABSTRACT

A photoacoustic wave measurement device includes: a thermal light source that irradiates a measurement target with infrared light that is modulated, in conjunction with heat generation corresponding to a modulation pattern; and a measurement unit that receives, in an airtight chamber, a photoacoustic wave emitted from the measurement target irradiated with the infrared light, and measures a pressure change in the airtight chamber by a pressure sensor.

### TECHNICAL FIELD

The present invention relates to a photoacoustic wave measurement device, a photoacoustic wave measurement system, and a thermal light source.

### BACKGROUND ART

A photoacoustic wave measurement device is known as a device for measuring a molecular concentration inside a measurement target. In a case where the measurement target is irradiated with modulated light, a photoacoustic wave (elastic wave) corresponding to a modulation pattern of the modulated light is generated when an internal molecule absorbs light and instantaneously thermally expands. The photoacoustic wave measurement device can detect the photoacoustic wave and determine a concentration of the molecule that absorbs the modulated light, according to the magnitude of the detected photoacoustic wave. As an example of such a photoacoustic wave measurement device, there is known a device that measures a pressure change in an airtight chamber that changes according to a photoacoustic wave using a pressure sensor and measures a molecular concentration of a measurement target (for example, Patent Literature 1).

### CITATION LIST

### Patent Literature

Patent Literature 1: JP2019-7824A

### SUMMARY OF INVENTION

### Technical Problem

According to the technique disclosed in Patent Literature 1, a laser light source is used as a light source for modulated light. When a laser light source is used to generate modulated light, a chopper device that performs light modulation is further required in addition to the laser light source. Therefore, a chopper device is required in addition to a relatively large laser light source, and there is a problem that the entire photoacoustic wave measurement device is increased in size.

The present invention has been made to solve such a problem, and an object thereof is to miniaturize a photoacoustic wave measurement device by using a thermal light source.

### Solution to Problem

A photoacoustic wave measurement device according to an aspect of the present invention includes: a thermal light source that irradiates a measurement target with infrared light that is modulated, in conjunction with heat generation corresponding to a modulation pattern; and a measurement unit that receives, in an airtight chamber, a photoacoustic wave emitted from the measurement target irradiated with the infrared light, and measures a pressure change in the airtight chamber by a pressure sensor.

A photoacoustic wave measurement system according to a further aspect of the present invention includes: a thermal light source that irradiates a measurement target with infrared light; a measurement unit that receives, in an airtight chamber, a photoacoustic wave emitted from the measurement target irradiated with the infrared light, and measures a pressure change in the airtight chamber by a pressure sensor; and a controller connected to the thermal light source and the measurement unit. The controller controls the thermal light source according to a modulation pattern to irradiate the measurement target with the infrared light that is modulated, and obtains a concentration of the measurement target according to the pressure change in the airtight chamber acquired by the measurement unit.

A thermal light source according to a further aspect of the present invention irradiates a measurement target with infrared light that is modulated, in conjunction with heat generation corresponding to a modulation pattern.

### Advantageous Effects of Invention

With the photoacoustic wave measurement device according to an aspect of the present invention, the thermal light source is used as the light source necessary for generating the photoacoustic wave used for measurement by the photoacoustic wave measurement device. The thermal light source can be miniaturized as compared with the laser light source. Since the temperature of the laser light source becomes high during irradiation with the laser light, ON and OFF of the irradiation with the laser light cannot be accurately switched, and it is necessary to further include a chopper portion for modulation. On the other hand, the thermal light source has a simple configuration, can be easily miniaturized, and is easily cooled while not generating heat, and therefore, modulation control may be performed only by turning on or off the current, and the chopper portion becomes unnecessary. By using such a thermal light source as the light source for infrared light, the photoacoustic wave measurement device can be miniaturized.

### BRIEF DESCRIPTION OF DRAWINGS

FIG. 1 is a schematic configuration diagram of a photoacoustic wave measurement device according to a first embodiment;

FIG. 2 is a sectional view of a sensor unit;

FIG. 3 is an exploded perspective view of the sensor unit;

FIG. 4 is a schematic configuration diagram of a thermal light source;

FIG. 5 is a graph showing properties of radiated light in a general heat source;

FIG. 6 is a graph showing luminance of irradiation light from the thermal light source;

FIG. 7 is a graph showing responsiveness of the thermal light source;

FIG. 8 is a graph showing measurement results by the photoacoustic wave measurement device;

FIG. 9 is a schematic configuration diagram of a photoacoustic wave measurement device according to a second embodiment;

FIG. 10 is a diagram illustrating an example of a variation of a configuration of a thermal light source in a third embodiment;

FIG. 11 is a diagram illustrating another example of the variation of the configuration of the thermal light source;

FIG. 12 is a diagram illustrating another example of a variation of a configuration of a photoacoustic wave measurement device;

FIG. 13 is a schematic configuration diagram of a photoacoustic wave measurement device according to a fourth embodiment; and

FIG. 14 is a schematic configuration diagram of a photoacoustic wave measurement device according to a fifth embodiment.

### DESCRIPTION OF EMBODIMENTS

Hereinafter, embodiments of the present invention will be described with reference to the drawings.

### First Embodiment

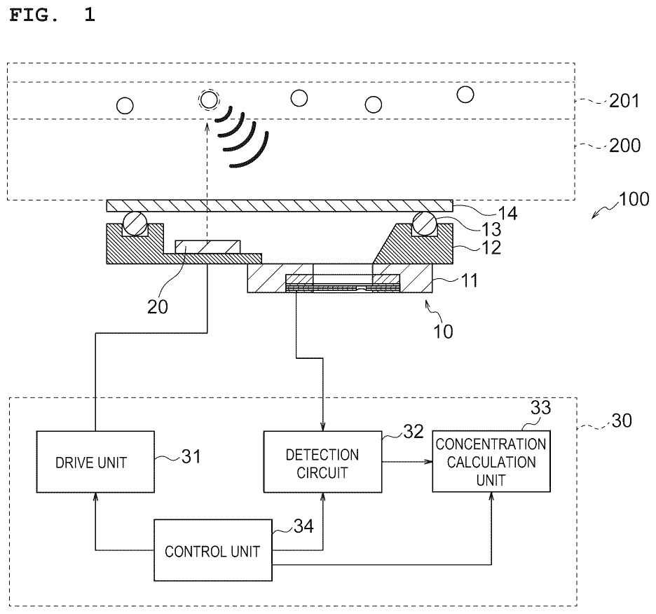

FIG. 1 is a schematic configuration diagram of a photoacoustic wave measurement device according to a first embodiment. In FIG. 1, a photoacoustic wave measurement device (hereinafter, simply referred to as a measurement device) 100 includes a measurement unit 10 that performs photoacoustic wave measurement, a thermal light source 20, and a circuit unit 30. The measurement device 100 can measure a concentration of a predetermined component present in a measurement target 200.

An outline of measurement on the concentration of the predetermined component in the measurement target 200 is as follows. In a case where the measurement target 200 is irradiated with light emitted from the thermal light source 20, a photoacoustic wave (elastic wave) is generated by a photoacoustic effect in molecules in the measurement target 200. The measurement unit 10 detects the photoacoustic wave generated from the measurement target 200. The circuit unit 30 calculates a concentration of a liquid measured in the measurement target 200 according to a change in intensity of the photoacoustic wave measured by the measurement unit 10.

In the example of the present embodiment, the measurement target 200 is a living body, and in a case where the measurement target 200 is irradiated with far-infrared light from the thermal light source 20, a photoacoustic wave is emitted from glucose in blood flowing through a blood vessel 201 in the measurement target 200. The photoacoustic wave is detected by the measurement unit 10, and a blood glucose concentration in the blood vessel 201 is calculated based on a detection result. Hereinafter, each configuration will be described in detail.

The circuit unit 30 includes a drive unit 31, a detection circuit 32, a concentration calculation unit 33, and a control unit 34 that collectively controls these units.

The thermal light source 20 outputs a modulated light of far-infrared light (wavelength of about 10 μm) toward the measurement target 200 under control of the drive unit 31. The thermal light source 20 is an electrical resistor as described later, and emits infrared light together with heat generation in response to energization. Modulation of far-infrared light may also be performed by controlling energization. Furthermore, light (for example, near-infrared light or mid-infrared light) corresponding to the temperature can be emitted by controlling the temperature of the thermal light source 20. In one measurement, a pulsed light is output a plurality of times, for example, about five times. Intensity-modulated light is not limited to a pulsed light in which output of the light is completely blocked, and the light may be modulated to reduce the intensity to a predetermined level (>0), for example.

In a case where the measurement target 200 is irradiated with the modulated light from the thermal light source 20 and the far-infrared modulated light reaches the blood vessel 201, glucose in the blood absorbs the far-infrared light and instantaneously expands to generate a photoacoustic wave (elastic wave). The photoacoustic wave generates a pressure change in an airtight chamber included in the measurement unit 10, and the pressure change is measured by the sensor unit 11. Here, since a larger photoacoustic wave is generated as the glucose concentration is higher, the concentration calculation unit 33 can calculate the blood glucose concentration according to the intensity of the photoacoustic wave. The concentration calculation unit 33 can remove (reduce) noise and determine the concentration with higher accuracy by synchronizing and associating a modulation period of the thermal light source 20 with the pressure change measured by the measurement unit 10.

The measurement unit 10 includes a sensor unit 11, a housing 12, a seal member 13, and a contact portion 14.

The housing 12 is a bottomed member with an upper portion opened and a space inside. The measurement unit 10 is fixed in a state where an end portion on a side surface of the housing 12 which is a bottomed member is pressed against the measurement target 200 via the contact portion 14. Further, the internal space of the housing 12 communicates with an opening provided in the sensor unit 11 and is integrated with a space between the housing 12 and the contact portion 14 to form an airtight chamber. The seal member 13 is provided between the housing 12 and the contact portion 14, so that the degree of adhesion between the housing 12 and the contact portion 14 is increased, and the airtightness of the space (airtight chamber) in the housing 12 is improved. An example of the seal member 13 is an O-ring, and is disposed in a groove portion provided in an end portion on the side surface of the housing 12. The seal member is also referred to as a sealing member.

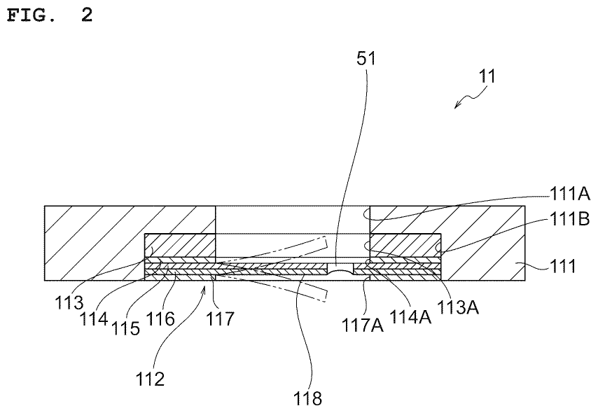

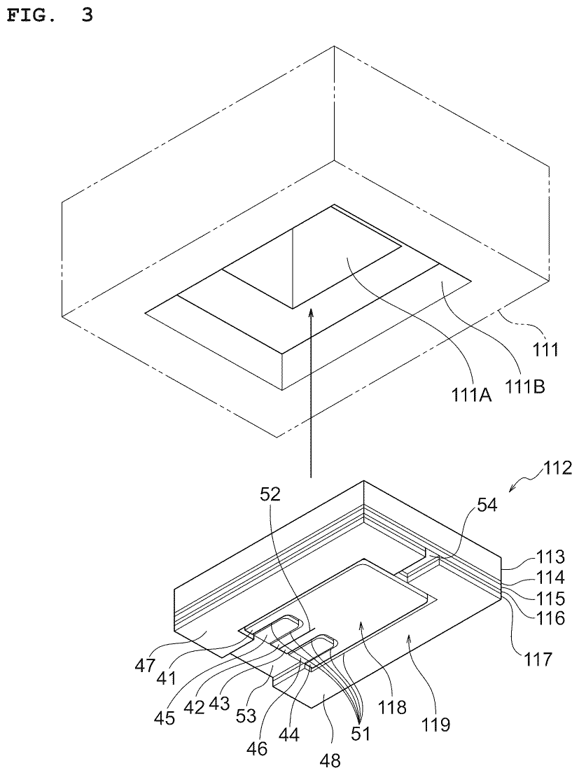

The intensity of the photoacoustic wave can be calculated by measuring, by the sensor unit 11, a change in air pressure in the airtight chamber configured in this manner. A structure of the sensor unit 11 will be described later with reference to FIGS. 2 and 3. In FIG. 1, the housing 12 is a bottomed member with an upper portion opened, but is not limited thereto. The housing 12 may have any structure as long as a space that may be an airtight chamber exists therein.

The contact portion 14 is made of a substance that has high transmittance for far-infrared light and is physically easily vibrated. The contact portion 14 is made of, for example, polyethylene having a thickness of 70 μm. Since a thin film of silicon or germanium has a property of physically easily vibrating, the contact portion 14 may be formed of a thin film of silicon or germanium having a thickness of 100 μm or less. The contact portion 14 is preferably made of a material that is displaced following displacement on the surface of the measurement target. Since the displacement due to the influence of the photoacoustic wave may occur even in the measurement target, the influence of the photoacoustic wave in the airtight chamber can be increased by forming the contact portion 14 with an elastic substance, and the measurement accuracy of the predetermined component can be improved.

FIG. 2 is an enlarged view of the sensor unit 11. As illustrated in an exploded perspective view of the sensor unit 11 in FIG. 3, in the sensor unit 11, a sensor substrate 112 is fitted and fixed to a fixing member 111 having a stepped opening inside.

Referring again to FIG. 2, the fixing member 111 includes an opening 111A provided on an airtight chamber side and having a relatively small opening area, and an opening 111B provided outside the measurement unit 10 and having a relatively large opening area. The openings 111A and 111B communicate with each other, and a step portion is formed therebetween. The sensor substrate 112 is fitted into the opening 111B and is fixed to the step portion of the fixing member 111 by locking.

The sensor substrate 112 is formed by laminating a silicon substrate 113, an insulating layer 114, a silicon layer 115, a piezoresistive layer 116, and a metal layer 117. The silicon substrate 113, the insulating layer 114, and the metal layer 117 include openings 113A, 114A, and 117A, respectively. The openings 113A, 114A, and 117A have approximately the same opening area as the opening 111A provided on the airtight chamber side of the fixing member 111.

The silicon layer 115 and the piezoresistive layer 116 are integrated, and one end portion (left side in FIG. 2) is sandwiched and fixed between the metal layer 117 and the silicon substrate 113 and the insulating layer 114. A groove portion 51 is provided, so that the other end portion of the silicon layer 115 and the piezoresistive layer 116 (right side in FIG. 2) becomes a free end without being fixed. Furthermore, as illustrated in FIG. 3, the silicon layer 115 and the piezoresistive layer 116 are configured such that a pressure receiving portion 118 at a central portion is rectangular, and are disposed inside a frame 119. The pressure receiving portion 118 is fixed to the frame 119 only at one side (left side in FIG. 2, left lower side in FIG. 3), and the other three sides are free ends that are not fixed to the frame 119. Therefore, the pressure receiving portion 118 is in a cantilever manner. A sensing portion having such a configuration of the pressure receiving portion 118 is generally referred to as a cantilever.

Referring again to FIG. 2, the pressure receiving portion 118 (silicon layer 115 and piezoresistive layer 116) in a cantilever manner is a plate-like member having a thickness of, for example, about 0.3 μm. As a result, the pressure receiving portion 118 is displaced (vibrated) in a vertical direction with respect to the opening according to an air pressure difference between the airtight chamber (openings 111A, 113A, and 114A) and the outside. The piezoresistive layer 116 has a property of increasing or decreasing a resistance value by compression or extension. Accordingly, a change of the airtight chamber caused by the photoacoustic wave emitted from the measurement target 200 can be detected by measuring the resistance value of the piezoresistive layer 116.

FIG. 3 is an exploded perspective view of the sensor unit 11. As illustrated in the drawing, in the sensor unit 11, the sensor substrate 112 is fitted and fixed to the opening 111B of the fixing member 111.

When the sensor substrate 112 is viewed in a lamination direction, the rectangular pressure receiving portion 118 is surrounded by the frame 119 and is in a cantilever manner fixed to the frame 119 only on one side (lower left side). As described above, the pressure receiving portion 118 is fixed to the frame 119 by integrally sandwiching the silicon layer 115 and the piezoresistive layer 116 between the metal layer 117 and the silicon substrate 113 and the insulating layer 114.

Furthermore, when attention is paid to one side (lower left side) where the pressure receiving portion 118 is fixed to the frame 119, the pressure receiving portion 118 is fixed to the frame 119 via four beam portions 41 to 44.

The beam portions 41 and 42 form a pair, and the beam portions 43 and 44 form a pair. The beam portions 41 and 44 are located at two ends, and the beam portions 42 and 43 are adjacent to each other via a slit groove portion 52. Between the paired beam portions 41 and 42 and between the paired beam portions 43 and 44, rectangular protruding portions 45 and 46 protruding from the frame 119 are respectively provided. The protruding portions 45 and 46 include the silicon layer 115 and the piezoresistive layer 116, and are formed integrally with the frame 119. The protruding portions 45 and 46 are separated from the pressure receiving portion 118 via the groove portion 51 on three sides other than a side in contact with the frame 119.

In this manner, the pressure receiving portion 118 is connected to the frame 119 via the beam portions 41 to 44 on one side (lower left side) on which the beam portions 41 to 44 are provided. At the same time, the pressure receiving portion 118 is separated, via the groove portion 51, from the frame 119 at three sides (lower right side, upper right side, and upper left side) where the beam portions 41 to 44 are not provided, and is also separated from the protruding portions 45 and 46. As described above, the slit groove portion 52 is provided between the beam portions 42 and 43. The groove portion 51 and the slit groove portion 52 are formed with a width of, for example, about 0.02 μm to 10 μm.

Furthermore, in the metal layer 117, an insulating groove portion 53 is provided between the beam portions 41 and 44 on a side (lower left side) where the pressure receiving portion 118 is fixed in a cantilever manner, and an insulating groove portion 54 is provided in a center portion on an opposite side (upper right side). The insulating groove portions 53 and 54 are provided to electrically insulate the inside of the metal layer 117, and accordingly, two electrodes 47 and 48 are formed.

The beam portion 41 of the piezoresistive layer 116 is connected to the electrode 47, and the beam portion 44 is connected to the electrode 48. The beam portions 42 and 43 are electrically insulated from the metal layer 117. In the piezoresistive layer 116, a conductive path is formed in a substantially U-shape connected by a path from the beam portion 41 at one end to the beam portion 44 at the other end through the pressure receiving portion 118. Therefore, the resistance value of the pressure receiving portion 118 can be calculated by measuring a current between the electrodes 47 and 48.

In this configuration, the pressure receiving portion 118 is elastically deformable around base ends of the beam portions 41 to 44 in a normal direction (upper-lower direction in drawing) of the surface. Since the beam portions 41 to 44 are curved due to deformation of the pressure receiving portion 118, the resistance value of the piezoresistive layer 116 increases or decreases. As a result, a change in the air pressure of the airtight chamber caused by the received photoacoustic wave can be measured by measuring a resistance between the electrodes 47 and 48.

A method of manufacturing the sensor unit 11 is as follows. First, a silicon on insulator (SOI) substrate in which the silicon substrate 113, the insulating layer 114, and the silicon layer 115 are laminated is prepared. Thicknesses of the silicon substrate 113, the insulating layer 114, and the silicon layer 115 are, for example, 300 μm, 0.4 μm, and 0.08 μm in this order. The silicon layer 115 of the SOI substrate is doped to form the piezoresistive layer 116 on a surface layer of the silicon layer 115. After the metal layer 117 serving as an electrode is formed on the piezoresistive layer 116, the metal layer 117, the piezoresistive layer 116, and the silicon layer 115 are sequentially etched to form the opening 117A, the groove portion 51, and the slit groove portion 52. Furthermore, the insulating groove portions 53 and 54 are provided by etching the metal layer 117, so that the electrodes 47 and 48 are formed. Finally, the silicon substrate 113 and the insulating layer 114 are etched from an opposite surface side to form the openings 113A and 114A. In this manner, the sensor substrate 112 including the pressure receiving portion 118 is formed.

The measurement unit 10 as a includes, reference resistance, a cantilever (not illustrated) having the same configuration as that of the cantilever included in the sensor unit 11. The detection circuit 32 compares a resistance value of the cantilever forming a part of an outer wall of the airtight chamber with a resistance value of the reference resistance to detect a change in the air pressure of the airtight chamber.

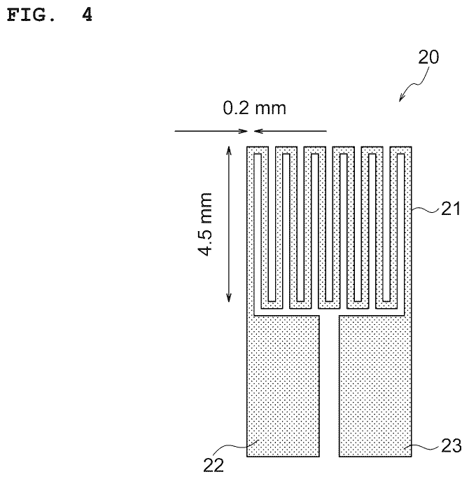

FIG. 4 is a schematic configuration diagram of the thermal light source 20. The thermal light source 20 is formed by finely patterning a copper foil formed on a glass composite material having a thickness of about 1 mm. In this drawing, a surface of the composite material provided with the copper foil is viewed from above. For example, the thickness of the copper foil is 50 μm, and on the composite material, an electrical resistor 21 provided in an upper region of the drawing and two electrodes 22 and 23 provided in a lower region of the drawing are provided. The thermal light source 20 is not limited to a copper foil, and may be made of tungsten. The thermal light source 20 can be made of a conductor such as metal.

In the example of this drawing, the electrical resistor 21 is patterned with a width of 0.2 mm and folded back 12 times every 4.5 mm. One ends of the electrodes 22 and 23 in an upper portion of the drawing are connected to both ends of the electrical resistor 21, and the other ends of the electrodes 22 and 23 in a lower portion of the drawing are connected to a power source (drive unit 31). The electrical resistor 21 has a relatively high resistance value, and in a case where electric power is applied to the electrodes 22 and 23, a current flows through the electrical resistor 21 and heat is generated. Furthermore, the thermal light source 20 is provided with a temperature sensor (not illustrated), and a temperature of the electrical resistor 21 acquired by the temperature sensor is input to the drive unit 31. The drive unit 31 can maintain the electrical resistor 21 at a desired temperature by performing feedback control using a measured temperature by the temperature sensor.

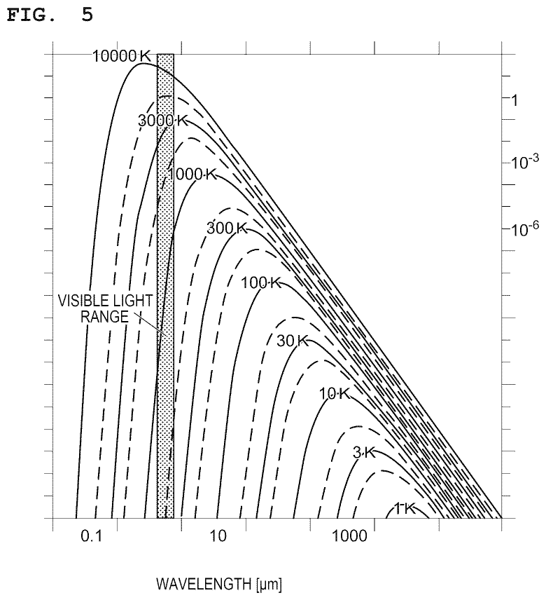

FIG. 5 is a graph showing a relation between a wavelength and a luminance (intensity) distribution of radiated light in a general heat source for each temperature. In this drawing, a radiation spectrum when the emissivity of the light from the heat source is 100% is shown, and a horizontal axis indicates the wavelength of the radiated light and a vertical axis indicates the luminance for each temperature of the heat source. The temperature in this drawing is shown using an absolute temperature (K).

As shown in this graph, the radiation spectrum varies depending on the temperature of the heat source. That is, light of a wavelength of a predetermined band is emitted according to the temperature of the heat source, and there is a wavelength at which the luminance becomes peak in the wavelength band. In the present embodiment, the electrical resistor 21 is controlled to 400 K, and far-infrared light is emitted. When the heat source is 400 K, far-infrared light of a wavelength of 9.3 μm is emitted at a peak intensity of 4.10⁻⁵ kW/cm²/μm. In this graph, the temperature is discretely shown, and the luminance distribution corresponding to 400 K is not shown, but the wavelength and luminance corresponding to 400 K can be generally understood.

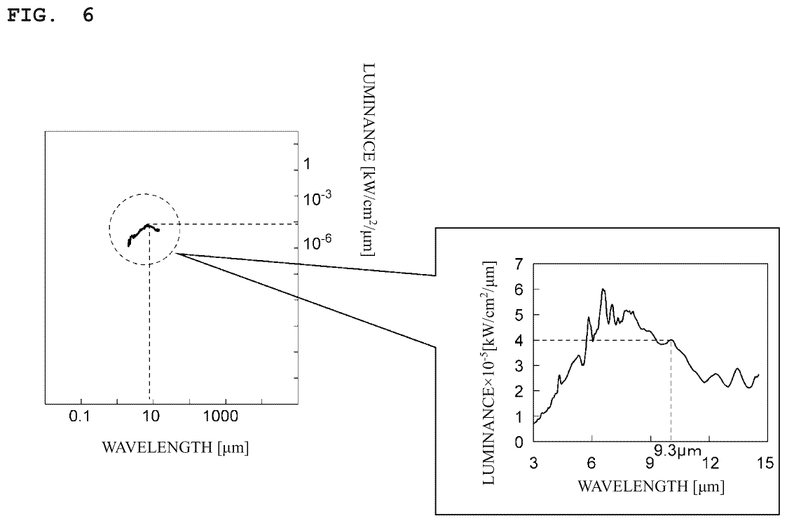

FIG. 6 is a graph showing the luminance of the irradiation light from the thermal light source 20 according to the present embodiment. This graph shows the radiated light when the thermal light source 20 generates heat at 400 K for a certain period of time. A horizontal axis indicates the wavelength, and a vertical axis indicates the luminance. In the graph on a left side, the wavelength and the luminance are shown by logarithm. The graph on a right side is an enlarged view of a part of the graph on the left side, and the luminance and the wavelength are shown by natural numbers. As shown in this graph, it is confirmed that when the thermal light source 20 is controlled under the above conditions, far-infrared light having a peak wavelength of 9.3 μm is emitted.

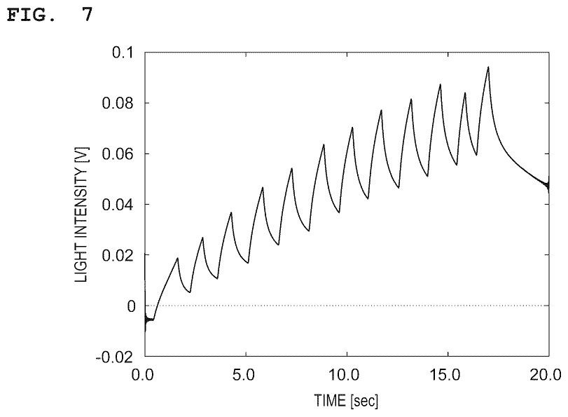

FIG. 7 is a graph showing the responsiveness of the thermal light source 20 according to the present embodiment. In this graph, a horizontal direction indicates time, and a vertical direction indicates the intensity of desired far-infrared light (wavelength of 9.3 μm). In the example of this graph, an initial resistance value of the electrical resistor 21 is 1.2 Ω, and a voltage of 2.5 V is periodically applied to the electrical resistor 21. Application and stop of the voltage is repeated at a cycle of about one second. It is confirmed that a current of 3.3 A flows while the voltage is applied, and it can be understood from the relation between the current and the voltage that the resistance value of the electrical resistor 21 under control is 0.76 Ω.

As shown in this graph, the light intensity of the far-infrared light emitted from the thermal light source 20 repeatedly increases and decreases according to periodic voltage application. By repeating the application and stop of the voltage over time, the light intensity increases as a whole. In the example of the graph, the modulation pattern is repeated at a cycle of about one second, but is not limited thereto. Any modulation pattern may be implemented by controlling the current in any pattern.

In this manner, the thermal light source 20 including the electrical resistor 21 is used as the light source for far-infrared light, so that it is possible to implement, with a simple configuration, the output of the far-infrared light having relatively high responsiveness of about one second interval. It is considered that the deterioration of the responsiveness of the thermal light source 20 is suppressed because the electrical resistor 21 is formed by finely patterning a copper foil, the cooling efficiency of the electrical resistor 21 is high due to such a configuration, and the electrical resistor 21 is sufficiently cooled while the application of the voltage is stopped.

In the present embodiment, an example is described in which far-infrared light is emitted from the thermal light source 20, but the present invention is not limited thereto. By controlling the temperature of the electrical resistor 21 to another temperature, the radiation spectrum of the irradiation light can be changed. Therefore, by controlling the temperature of the thermal light source 20, it is possible to increase the intensity of light of another wavelength from the thermal light source 20, for example, near-infrared light or mid-infrared light.

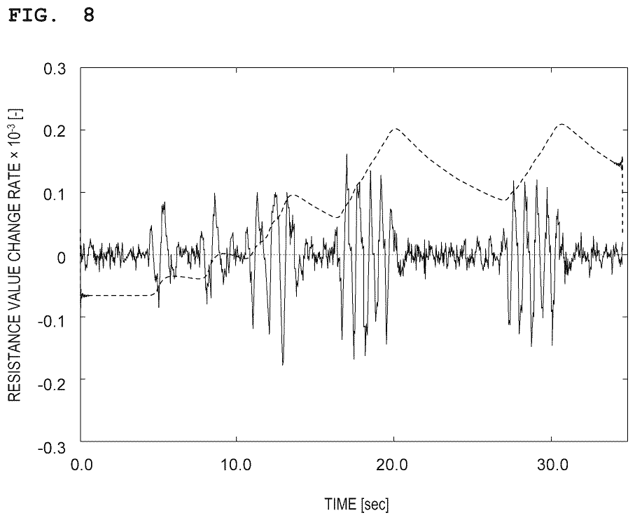

FIG. 8 is a graph showing measurement results by the measurement device 100. According to this graph, a resistance value change rate of the sensor unit 11 is shown by a solid line, and the intensity of the irradiation light from the thermal light source 20 is shown by a broken line. The resistance value change rate indicates a change rate of the resistance value of the sensor unit 11 (cantilever) provided in the airtight chamber with respect to the reference resistance.

In the example of this graph, the measurement target 200 is a glucose solution, and the electrical resistor 21 is controlled to 400 K. By repeating application and stop of a current of 2.5 A to the electrical resistor 21, the irradiation intensity of the far-infrared light from the thermal light source 20 repeatedly increases and decreases, and vibrates in a constant intensity range after a predetermined period of time elapses. An increase or decrease in the resistance value change rate of the sensor unit 11 is detected according to a change in the irradiation intensity of the far-infrared light.

These results indicate that, even when the thermal light source 20 including the electrical resistor 21 is used to emit far-infrared light, the pressure in the airtight chamber of the measurement unit 10 is changed by the photoacoustic wave emitted from the glucose solution, and the pressure change can be measured by the cantilever. As a result, the light source can be miniaturized and the entire measurement device 100 can be miniaturized as compared with the case where a laser light source is used.

Furthermore, measurement on the concentration of the glucose solution by the concentration calculation unit 33 is performed by synchronizing a change pattern of the pressure change rate by the sensor unit 11 with the modulation pattern of the thermal light source 20. When modulation is performed using a chopper portion, since overhead occurs in an operation of the chopper portion, it is necessary to perform synchronization in consideration of an overhead time. On the other hand, when the thermal light source 20 is used, since the modulation pattern is determined in accordance with an energization timing of the electrical resistor 21 by the drive unit 31, synchronization between the modulation pattern of the far-infrared light and the measurement result by the detection circuit 32 is facilitated.

In the present embodiment, an example is described in which far-infrared light is emitted from the thermal light source 20, but the present invention is not limited thereto. The measurement target 200 may be irradiated with near-infrared light or mid-infrared light by changing the control temperature of the thermal light source 20.

In the present embodiment, an example is described in which one measurement unit 10 is provided, but the present invention is not limited thereto, and a plurality of measurement units may be provided. For example, a generation position of the photoacoustic wave can be specified by overlapping frequency bands of the measurable pressure change of the plurality of measurement units 10. The frequency bands of the measurable pressure change of the plurality of measurement units 10 are different from one another, so that the vibration corresponding to the measurement target can be detected, and therefore, the concentrations of the plurality of measurement targets can be simultaneously measured.

In the present embodiment, the thermal light source 20 is used in the photoacoustic wave measurement device 100, but the present invention is not limited thereto. The thermal light source 20 is controlled according to the modulation pattern, so that the modulated infrared light can be emitted to any measurement target.

### Second Embodiment

In the first embodiment, the contact portion 14 is formed of a single member, but the present invention is not limited thereto. In the present embodiment, an example in which a part of the contact portion 14 is formed of a different member will be described.

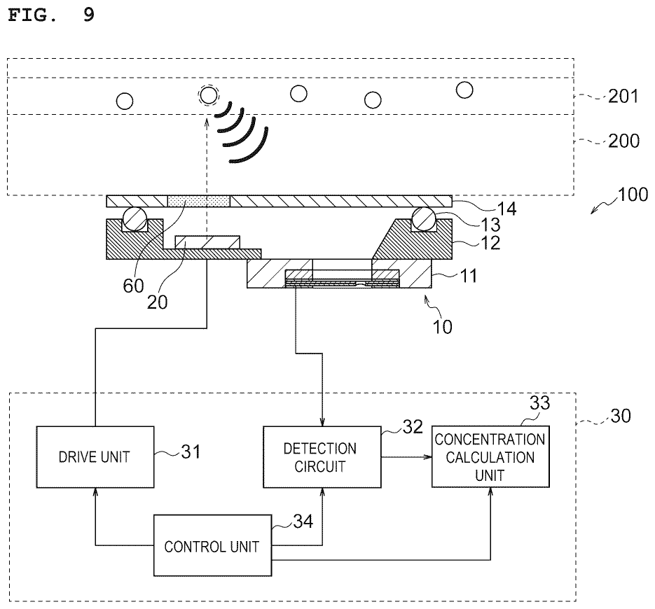

FIG. 9 is a schematic configuration diagram of the measurement device 100 according to a second embodiment, and corresponds to FIG. 1. According to this drawing, as compared with the measurement device 100 according to the first embodiment, a part of the contact portion 14 is formed of another member, thereby forming a window portion 60. In the contact portion 14, a portion other than the window portion 60 is made of a substance that is physically easily vibrated, and the window portion 60 is made of a substance having high transmittance of far-infrared light. For example, the portion of the contact portion 14 other than the window portion 60 is made of polyethylene, and the window portion 60 is made of a thin film (for example, thickness of 100 μm or less) of silicon or germanium. In this manner, the intensity of far-infrared light emitted to the measurement target 200 can be increased, and therefore, it is expected that a stronger photoacoustic wave is generated from the measurement target 200. As a result, the pressure change in the airtight chamber increases, and the measurement accuracy of the measurement device 100 can be improved.

In another aspect, the window portion 60 may be made of a material that is hardly affected by far-infrared light, for example, without alteration by irradiation with far-infrared light or alteration by absorption of far-infrared light. The window portion 60 may optionally have such a property, and may have at least one of high transmittance, high heat resistance, and high durability of far-infrared light. The window portion 60 is made of, for example, a thin film of silicon or germanium which is resistant to heat. As an example, germanium has a thickness of about 1 mm and is hardly deformed. In general, in a case where the thickness of the material is increased, it is difficult to transmit the far-infrared light, and therefore, the material and the thickness of the window portion 60 are determined by design so that a transmission amount of the far-infrared light is sufficient. When the far-infrared light is sufficiently transmitted, it is preferable that the thickness is large because the influence of the outside air pressure on the airtight chamber can be suppressed. Alternatively, the window portion 60 may be made of polyethylene.

The thermal light source 20 may be provided outside the airtight chamber. In such a configuration, the measurement unit 10 is provided between the thermal light source 20 and the measurement target 200. In a light guiding path for the irradiation light from the thermal light source 20, a window portion made of a similar member having high transmittance of far-infrared light is provided in a wall portion (lower side in drawing) which partitions the airtight chamber from the outside, in addition to a part of the contact portion 14. With such a configuration, even in a configuration in which the thermal light source 20 is outside the measurement unit 10 and the measurement target 200 is irradiated with far-infrared light via the measurement unit 10, the intensity of the far-infrared light emitted to the measurement target 200 is increased, and the measurement accuracy of the measurement device 100 can be improved.

### Third Embodiment

In the third embodiment, variations of the configuration of the thermal light source 20 will be described.

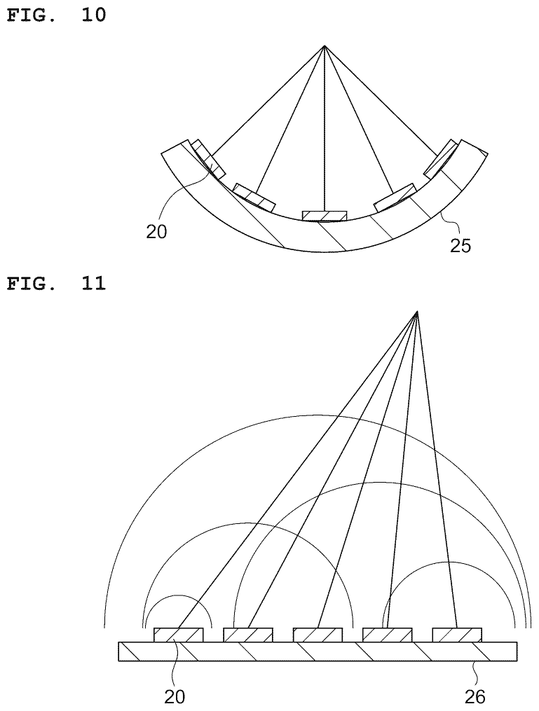

FIG. 10 is a diagram illustrating an example of an arrangement of the thermal light source 20. According to the drawing, a support portion 25 is curved in a concave paraboloid shape, and a plurality of thermal light sources 20 are provided on a surface of the support portion 25. The thermal light sources 20 are arranged on the support portion 25 whose surface is in a paraboloid shape, so that the far-infrared light emitted from the plurality of thermal light sources 20 is concentrated at a center of the paraboloid and the intensity is increased. For example, the support portion 25 is configured and disposed in the measurement device 100 such that the center of the paraboloid becomes the measurement target 200 (blood vessel 201), so that the intensity of the far-infrared light emitted to the measurement target 200 can be increased, and therefore, the measurement accuracy of the measurement device 100 can be improved. The support portion 25 is not limited to a paraboloid, and may include a light condensing surface that is capable of condensing, on the measurement target 200, irradiation light from the thermal light sources 20. The light condensing surface may be, for example, a mirror configured by a curved mirror surface. Furthermore, the light condensing surface is not limited to a light condensing surface having a shape and aspect such as a paraboloid or a curved mirror, and may include any light condensing mechanism. For example, as long as the plurality of thermal light sources 20 are arranged on a light condensing surface and a combined wave of infrared light emitted from the thermal light sources 20 is condensed on the measurement target 200, the light condensing mechanism can take any shape and aspect.

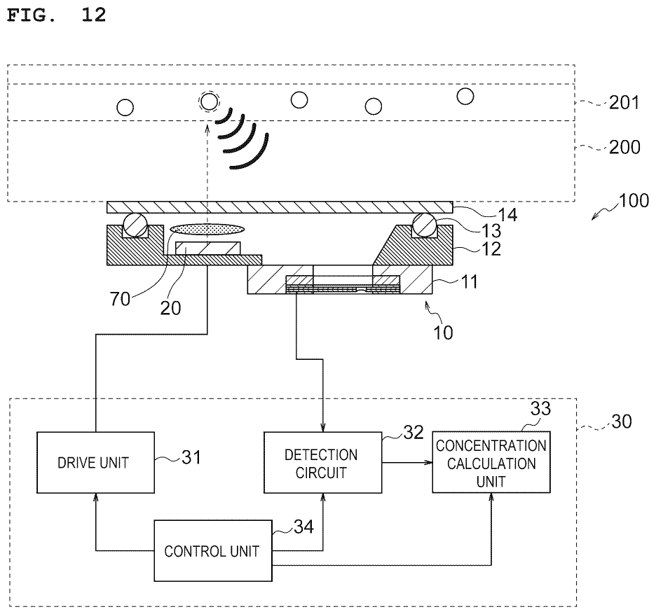

FIG. 11 is a diagram illustrating another example of the arrangement of the thermal light sources 20. According to this drawing, the plurality of thermal light sources 20 are provided on a plate-like support portion 26, and each of the thermal light sources 20 is modulated according to each modulation pattern. The thermal light sources 20 are subjected to modulation control with a difference in phase so that far-infrared light emitted from the plurality of thermal light sources 20 is concentrated on the measurement target 200 (blood vessel 201). Even with such a configuration, the intensity of the far-infrared light emitted to the inside of the measurement target 200 can be increased, and therefore, the measurement accuracy of the measurement device 100 can be improved.

Alternatively, a phased array having directivity may include the plurality of thermal light sources 20. Specifically, the directivity may be given to the emitted far-infrared light by providing a phase shifter and an antenna element for the plurality of thermal light sources 20 arranged on a plane and controlling a phase amount of the modulation pattern of the far-infrared light.

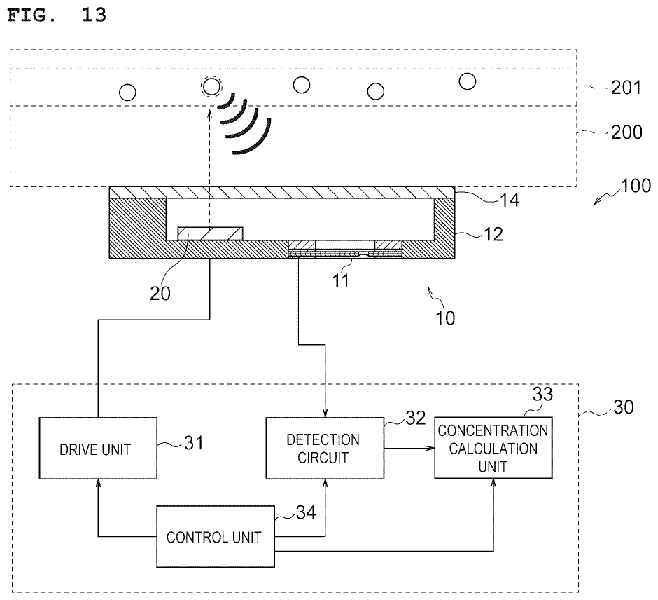

FIG. 12 illustrates another aspect relating to the thermal light source 20 of the measurement device 100. According to this drawing, a filter 70 is provided in the light guiding path for far-infrared light from the thermal light source 20 between the thermal light source 20 and the measurement target 200. The filter 70 has a property of transmitting only far-infrared light of a predetermined wavelength. By providing such a filter 70, the measurement target 200 can be irradiated with far-infrared light of a predetermined wavelength. Furthermore, the filter 70 may be in a lens shape so that the far-infrared light is concentrated on the blood vessel 201 of the measurement target 200, and may be disposed so that a focal point thereof is within the measurement target 200.

In this manner, the thermal light source 20 is capable of blocking condensed far-infrared light and light of a wavelength other than the predetermined wavelength, so that the measurement accuracy of the measurement device 100 can be improved. Furthermore, the temperature of the thermal light source 20 is controlled, so that the intensity of the infrared light of a desired wavelength with respect to the measurement target 200 can be controlled via the filter 70. In a case where the temperature of the thermal light source 20 is changed, emission of different spectra is implemented. In such a case, when a plurality of filters 70 that transmit light of different wavelengths are provided, it is possible to change the intensity of the infrared light of a plurality of wavelengths with which the measurement target 200 is irradiated. In this manner, the wavelength and the intensity of the infrared light reaching the measurement target 200 can be controlled. The temperature of the thermal light source 20 is changed over time, so that it is possible to irradiate the measurement target 200 with infrared light of various wavelengths and intensities by one control. The filter 70 may be one, and in this case, the intensity of infrared light of a predetermined wavelength can be changed by changing the temperature of the thermal light source 20.

### Fourth Embodiment

In the first to third embodiments, examples are described in which in the measurement unit 10, the sensor unit 11 is attached to a wall surface of the housing 12, but the present invention is not limited thereto. In a fourth embodiment, an example in which the sensor unit 11 constitutes a part of the wall surface of the housing 12 will be described.

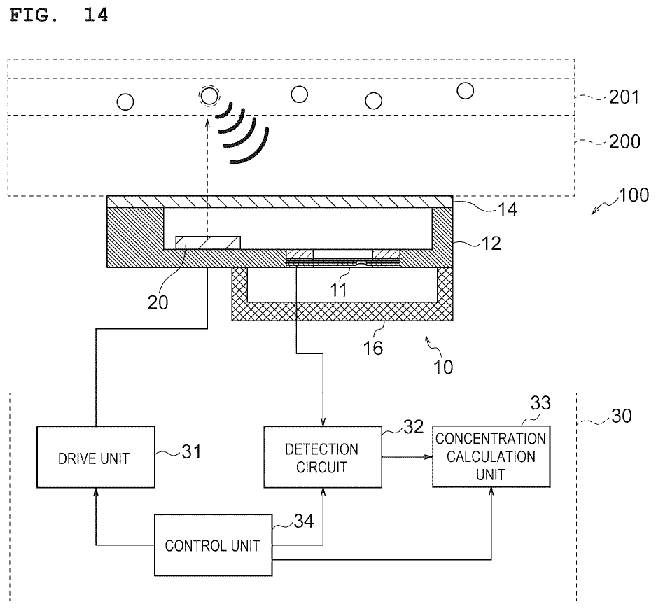

FIG. 13 is a schematic configuration diagram of the measurement device 100 according to the fourth embodiment. As compared with the measurement device 100 according to the first embodiment illustrated in FIG. 1, the seal member 13 is eliminated, and the shape of the housing 12 is changed. Similarly to the other embodiments, the housing 12 constitutes an airtight chamber. Furthermore, the sensor unit 11 does not include the fixing member 111, and the sensor substrate 112 constitutes a part of a wall surface of the housing 12 without being fitted into the fixing member 111.

Even with such a configuration, a change in air pressure caused by the photoacoustic wave (elastic wave) in the airtight chamber is measured by the sensor unit 11. As a result, the change in the air pressure of the airtight chamber can be easily measured, and therefore, the measurement accuracy of the measurement device 100 can be improved.

### Fifth Embodiment

In the fourth embodiment, in the measurement unit 10, the sensor unit 11 constitutes a part of a wall surface of the housing 12, and the sensor unit 11 separates the airtight chamber from the outside, but the present invention is not limited thereto. In a fifth embodiment, an example in which a closed space is further provided on a side opposite to the airtight chamber of the sensor unit 11 (cantilever) will be described.

FIG. 14 is a schematic configuration diagram of the measurement device 100 according to the fifth embodiment. As compared with the measurement device 100 according to the fourth embodiment illustrated in FIG. 13, a housing 16 is further provided on a side opposite to a measurement target 200 side of the sensor unit 11 (cantilever). Similarly to the housing 12, the inside of the housing 16 is closed to form a closed space.

The sensor unit 11 detects the pressure of the airtight chamber affected by the photoacoustic wave emitted from the measurement target 200, and this is performed by measuring a displacement caused by an air pressure difference in a space facing both directions of the displacement (vertical direction in drawing) by a sensing portion (cantilever). That is, in the first to fourth embodiments, the sensor unit 11 measures the displacement of the sensing portion caused by a pressure difference between the airtight chamber and the outside air. On the other hand, in the fifth embodiment, the magnitude of the photoacoustic wave is measured by measuring the displacement of the sensing portion caused by the pressure difference between the airtight chamber on an upper side in the drawing which is close to the measurement target 200 and is easily affected by the photoacoustic wave and the closed space on a lower side in the drawing which is far from the measurement target 200 and is hardly affected by the photoacoustic wave. The closed space is provided on a side opposite to the airtight chamber of the sensor unit 11, so that the sensing portion is hardly affected by the surroundings than when the sensing portion is in directly contact with the outside air, and as a result, the measurement accuracy of the measurement device 100 can be improved. In the present embodiment, the airtight chamber in the housing 12 and the closed space in the housing 16, which are separated by the sensor unit 11, have different opening areas and different volumes, but the present invention is not limited thereto. Both the airtight chamber and the closed space may have any size.

Various embodiments and modifications may be made without departing from the broad spirit and scope of the present invention. The above-described embodiments are for describing the present invention, and do not limit the scope of the present invention. That is, the scope of the present invention is indicated by the claims rather than the embodiments. Various modifications made within the scope of the claims and within the meaning of the invention equivalent thereto are regarded as within the scope of the present invention.

### REFERENCE SIGN LIST

10 measurement unit
11 sensor unit
12, 16 housing
13 seal member
14 contact portion
20 thermal light source
21 electrical resistor
22, 23 electrode
30 circuit unit
60 window portion
70 filter
100 photoacoustic wave measurement device
200 measurement target
201 blood vessel

1. A photoacoustic wave measurement device comprising:
a thermal light source that irradiates a measurement target with infrared light that is modulated, in conjunction with heat generation corresponding to a modulation pattern; and
a measurement unit that receives, in an airtight chamber, a photoacoustic wave emitted from the measurement target irradiated with the infrared light, and measures a pressure change in the airtight chamber by a pressure sensor.

2. The photoacoustic wave measurement device according to claim 1, wherein
the thermal light source is an electrical resistor that generates heat when a current flows therethrough.

3. The photoacoustic wave measurement device according to claim 2, wherein
the electrical resistor is formed by patterning a conductor.

4. The photoacoustic wave measurement device according to claim 1, wherein
the thermal light source is controlled to a temperature corresponding to a desired radiation spectrum.

5. The photoacoustic wave measurement device according to claim 4, wherein
the thermal light source includes a temperature sensor, and is controlled to a temperature corresponding to the desired radiation spectrum by feedback control using a temperature acquired by the temperature sensor.

6. The photoacoustic wave measurement device according to claim 4, further comprising:
a filter that transmits light of a desired wavelength between the thermal light source and the measurement target.

7. The photoacoustic wave measurement device according to claim 1, further comprising:
a light condensing mechanism that condenses the infrared light on the measurement target between the thermal light source and the measurement target.

8. The photoacoustic wave measurement device according to claim 7, wherein
a plurality of thermal light sources including the thermal light source are provided, and
the light condensing mechanism is a phased array structure in which a phase amount of the modulation pattern of the plurality of thermal light sources is controlled to impart directivity to the infrared light emitted from the plurality of thermal light sources.

9. The photoacoustic wave measurement device according to claim 1, wherein
the thermal light source is provided in the airtight chamber.

10. The photoacoustic wave measurement device according to claim 9, wherein
the airtight chamber includes a window portion in a portion to be directly irradiated with the infrared light from the thermal light source, and
the window portion is made of a material having high transmittance of the infrared light.

11. The photoacoustic wave measurement device according to claim 1, further comprising:
outside the pressure sensor on a side opposite to the airtight chamber, a closed space that is separated from outside air.

12. A photoacoustic wave measurement system comprising:
a thermal light source that irradiates a measurement target with infrared light;
a measurement unit that receives, in an airtight chamber, a photoacoustic wave emitted from the measurement target irradiated with the infrared light, and measures a pressure change in the airtight chamber by a pressure sensor; and
a controller connected to the thermal light source and the measurement unit, wherein
the controller
controls the thermal light source according to a modulation pattern to irradiate the measurement target with the infrared light that is modulated, and
obtains a concentration of the measurement target according to the pressure change in the airtight chamber acquired by the measurement unit.

13. The photoacoustic wave measurement system according to claim 12, further comprising:
a filter that transmits light of a desired wavelength corresponding to the measurement target and is provided in a path from the thermal light source to the measurement target, wherein
the controller changes a temperature of the thermal light source to change an intensity of the infrared light of the desired wavelength with respect to the measurement target.

14. A thermal light source for irradiating a measurement target with infrared light that is modulated, in conjunction with heat generation corresponding to a modulation pattern.
